(19) United States
(12) Patent Application Publication
(10) Pub. No.: US 2008/0213396 A1
(43) Pub. Date: Sep. 4, 2008

(54) Copper Lowering Treatment Of Cardiac Disease

(75) Inventor: George J. Brewer, Ann Arbor, MI (US)

Correspondence Address: Casimir Jones, S.C., 440 Science Drive, Suite 203, Madison, WI, 53711, US

(73) Assignee: The Regents of the University of Michigan, Ann Arbor, MI (US)

(21) Appl. No.: 11/970,607

(22) Filed: Jan. 8, 2008

Related U.S. Application Data
(60) Provisional application No. 60/879,311, filed on Jan. 8, 2007.

(51) Int. Cl. A61K 33/30; A61K 33/24; A61K 31/19; A61K 31/47; A61K 31/704; A61P 9/00

(52) U.S. Cl. 424/641; 424/646; 514/562; 514/311; 514/34

(57) ABSTRACT

The present invention relates generally to the field of prophylaxis and therapy for cardiac fibrosis. In particular, the present invention is related to agents that can bind or complex copper, and to the use of these agents in the prevention and treatment of cardiac damage caused by chemotherapeutic and pharmaceutical agents.

This application claims priority to provisional patent application Ser. No. 60/879,311, filed Jan. 8, 2007, which is herein incorporated by reference in its entirety.

### FIELD OF THE INVENTION

The present invention relates generally to the field of prophylaxis and therapy for cardiac fibrosis. In particular, the present invention is related to agents that can bind or complex copper, and to the use of these agents in the prevention and treatment of cardiac damage caused by chemotherapeutic and pharmaceutical agents.

### BACKGROUND

Cardiac fibrosis is a hallmark of heart disease and is the result of a variety of structural changes that occur after pathological stimuli to the cardiovascular system (Judgutt B I (2003) Curr Drug Targets Cardiovasc Haematol Disord 3:1-30). The fibrosis in heart disease is characterized by a disproportionate accumulation of fibrillar collagen that occurs after myocyte death, inflammation, hypertrophy, and stimulation by a number of hormones, cytokines, and growth factors (See e.g., Weber K T (1989) J Am Coll Cardiol 13:1637-1652); Bishop J E, Lindahl G (1999) Cardiovasc Res 42:27-44; Lijnen P J). The proximal effector cells in this process are fibroblasts, which when activated to become myofibroblasts produce an excessive amount of collagen in response to inflammatory mediators, such as TGF-13 (Petrov W V, Fagard R H (2000) Mol Genet Metab 71:418-435) and angiotensin II (Ang II) (Lijnen P J, Petrov V V, Fagard R H (2000) Methods Find Exp Clin Pharmacol 22:709-723). Cardiac fibroblasts are the predominant source of synthesis of interstitial proteins and other myocardial components which have been implicated in heart failure by their effects on diastolic function and, indirectly, by effects on cardiac myocytes to cause or potentiate systolic dysfunction (Hess et al, Circ., 63:360-371 (1981); Villari et al, Am J. Cardiol., 69:927-934 (1992); Villari et al, JACC, 22:1477-1484 (1993); Brilla et al, Circ. Res., 69:107-115 (1991); and Sabbah et al, Mol. & Cell Biochem., 147:29-34 (1995)).

Acute ischemic damage to the heart is associated with a progression to congestive heart failure. In addition to the original damage, the process of remodelling results in an even greater scarring response than necessary to mitigate the original damage, and this remodelling takes place over many months following an ischemic event. The additional scarring leads to a compensatory response in the remaining tissue and an eventual hypertrophy and chronic failure.

Chronic damage leading to myocardial fibrosis is also associated with heart failure. Myocardial fibrosis occurs when the heart becomes abnormally enlarged, thickened and/or stiffened due to fibrosis. This condition is generally a progressive one and may be caused by a wide range of conditions, including hypertension, chronic diseases, alcoholism, viral diseases, and others. An affected heart may grow larger either by dilatation, hypertrophy, or both. Additionally, the fibrosis associated with cardiomyopathies leads to a lessening in function of the tissue and decreased contractility.

Hypertension affects 15-20% of the adult population leading to structural remodeling of the left ventricular (LV) myocardium and eventually heart failure. Several studies have focused on the contribution of the extracellular matrix to cardiac diastole and systole function. Tyagi S C et al. J Cell Physiol. (1996) 167:137-147. It is now well established that the fibrillar collagen network in the ECM is integral in providing the structural support for cardiomyocytes and coronary vessels, as it imparts the myocardium with physical properties and influences ventricular diastolic and systolic function. A pathological stimulus to this network leads to the development of cardiac fibrosis, an integral characteristic of hypertensive heart disease.

Doxorubicin hydrochloride (HCl) is an antineoplastic agent which is highly effective and has a broad spectrum of activity against many forms of cancer. It is the most widely used antineoplastic in many countries including the USA and Western Europe. Doxorubicin HCl is often included in multi-drug regimens and has become the mainstay of chemotherapy. This drug was originally developed and marketed in North America by Adria Laboratories, a Division of Erbamont Inc., under the trademark “Adriamycin.”

A serious side effect which may result from cumulative doses of doxorubicin HCl exceeding 550 mg/m² is irreversible myocardial toxicity with delayed congestive heart failure often unresponsive to cardiac support therapy. This toxicity may occur at lower cumulative doses in patients with prior mediastinal irradiation or on concurrent cyclophosphamide therapy.

Clinical studies have shown that the compound (S)-(+)-bis-4,4′-(1-methyl-1,2-ethanediyl)2,6-piperazinedione (dexrazoxane, formerly referred to as ADR-529, formerly referred to as ICRF-187 and marketed under the tradename ZINECARD®), is a cardioprotective agent which when administered contemporaneously with doxorubicin HCL and other antineoplastic anthracyclines such as adriamycin, can reduce or prevent the myocardial toxicity resulting from administration of doxorubicin HCl. (See Green et al, PROC. ASCO, 1987 6:28). Other highly desirable properties of ADR-529 are activity as a divalent cation chelating agent, sensitizer to ionizing radiation, anti-metastic agent and synergistic agent with anthracyclines in terms of anti-tumor effect.

Dexrazoxane has its own associated toxicities which recommend against its use in a pre-treatment fashion. In addition, dexrazoxane does not fully protect all patients but rather permits higher cumulative doses of anthracyclines to be administered to cancer patients. Because dexrazoxane requires intravenous administration as described in U.S. Pat. Nos. 4,963,551 and 5,242,901, dexrazoxane must be administered intravenously contemporaneously with anthracyclines. The FDA product label of ZINECARD recommends against administering doxrazoxane prior to anthracycline administration. Its use after anthracycline administration has not been tested in a controlled fashion and its repeated and/or chronic use is impractical due to its requirement of i.v. administration. The mechanism of action of dexrazoxane, a derivative of the chelator EDTA, is believed to due to its ability to chelate iron and reduce free radical damage to the heart. The iron chelator, desferoxamine (DFO) had been used prior to the introduction of dexrazoxane further supporting this hypothesis.

Antracycline induced toxicity is not immediate but instead generally becomes evident several days or weeks following exposure. Its effects are also cumulative such that patients having received higher previous doses are more susceptible to the myocardial fibrosis and, ultimately, congestive heart failure associated with anthracyclin induced myocardial fibrosis. Clearly what is needed is an alternative or adjunct therapy that can be used either alone or together with and if necessary chronically by means of convenient oral administration to permit higher cumulative doses of anthracyclines.

### SUMMARY OF THE INVENTION

The present invention relates generally to the field of prophylaxis and therapy for cardiac fibrosis. In particular, the present invention is related to agents that can bind or complex copper, and to the use of these agents in the prevention and treatment of cardiac damage caused by chemotherapeutic and pharmaceutical agents.

Accordingly, in some embodiments, the present invention provides a method of treating or preventing anthracycline induced myocardial fibrosis comprising administering a copper lowering agent to a subject under conditions such that anthracycline induced myocardial fibrosis is prevented or reduced. In some embodiments, the copper lowering agent is a thiomolybdate (e.g., tetrathiomolybdate). In some embodiments, the copper lowering agent is zinc, trientine, d-penicillamine, clioquinoil or tetrathiotungstate. In some embodiments, the copper lowering agent is administered prior to said anthracycline, contemporaneously with said anthracycline, or after said anthracycline. In some embodiments, the copper lowering agent is administered orally or parenterally.

In further embodiments, the present invention provides a method of treating or preventing non-ischemic or ischemic dilated cardiomyopathy having confirmed myocardial fibrosis, comprising administering a copper lowering agent under conditions such that symptoms of dilated cardiomyopathy are prevented or reduced. In some embodiments, the copper lowering agent is tetrathiomolybdate, trientine or zinc.

### DESCRIPTION OF THE FIGURES

FIG. 1 shows the plasma Cp levels in the three groups of mice that received doxorubicin (DXR) and one control group that did not receive DXR.

FIG. 2 shows the troponin I plasma levels in the four groups of mice 4 days after DXR.

FIG. 3 shows the creatinine kinase (CK)-MB plasma levels in the four groups of mice 4 days after DXR.

FIG. 4 shows the lactic dehydrogenase (LDH) plasma levels in the four groups of mice 4 days after DXR.

FIG. 5 shows treatment of myocardial fibrosis. FIG. 5A is a bar graph of the results of a comparative study of trientine in the doxorubicin heart model. FIGS. 5B and 5C show the lack of effect on LDH and CK-M. FIG. 5D shows a statistically insignificant effect of trientine on troponin I.

FIG. 6 shows the LDH plasma levels in the four groups of mice.

FIG. 7 shows the troponin I plasma levels in the four groups of mice.

### DEFINITIONS

To facilitate an understanding of the present invention, a number of abbreviations, terms and phrases as used herein are defined below.

The term “fibrotic” is used to refer to pertaining to or characterized by fibrosis. Fibrosis in disease or response to injury is the dysregulated excessive formation of fibrous tissue as a reactive process, as opposed to formation of fibrous tissue as a normal constituent of an organ or tissue, or as a part of normal repair of tissue.

The term “disease” refers to an interruption, cessation, or disorder of body function, systems, or organs. The term “disease” includes responses to injuries, especially if such responses are excessive. The term “condition” is used to refer to a disease or a response to injury. An “inflammatory disease” refers to a disease caused by or resulting from or resulting in inflammation. A “fibrotic disease” refers to a disease caused by or resulting from or resulting in fibrosis. A disease may include a response to injury, especially where the response is excessive, does not heal normally, and/or produces symptoms that excessively interfere with normal activities of an individual (where excessive is characterized as the degree of interference, or the length of the interference).

The term “injury” refers to damage or wound of trauma. A response to injury may be inflammation and/or fibrosis.

The term “anti-fibrotic” is used to refer to an effect or compound which has an effect of preventing, inhibiting, alleviating or decreasing fibrosis or components of the fibrotic reaction, either completely or partially.

The term “biocompatible” refers to compositions comprised of natural or synthetic materials, in any suitable combination, that remain substantially biologically unreactive in a subject or patient. The term “substantially unreactive” means that any response observed in a subject or patient is a subclinical response, i.e., a response that does not rise to a level necessary for therapy.

The term “biologically active agent” or “therapeutic agent” refers to an agent that possesses an activity or property capable of affecting or effecting a biochemical function, such as a structural (for example, binding ability) or regulatory activity or a reaction. Biochemical functions include but are not limited to physiological, genetic, cellular, tissue, and organismal activities. Moreover, as used herein, the term “agent” refers to biologically active agents and therapeutic agents, except where noted otherwise. Biological activities include activities associated with biological reactions or events in a subject or patient; preferably such activities can be detected, monitored, characterized, or measured.

The term “endogenous copper level” refers to the total amount of copper in the body of a patient; this amount includes both tissue and fluid amounts. The amount of copper in the body can also be divided into the amounts of available and amounts of unavailable copper. The “copper status” of a patient refers to the amount of available copper. Copper status is determined in the blood of a healthy individual, for example, by the concurrent measurement of plasma copper and ceruloplasmin. Normal plasma copper is present in two primary pools. Most plasma copper in normal individuals is part of the ceruloplasmin molecule. This copper is essentially unavailable for ready exchange with cells. Another pool of copper is more loosely bound to albumin and small molecules, such as amino acids. This latter pool of copper is readily available for cellular uptake. When tetrathiomolybdate (TM) enters the blood it complexes with the available copper, and renders it, like ceruloplasmin copper, unavailable for cellular uptake. In TM treated patients, copper status can be determined by measuring plasma ceruloplasmin alone. As the level of available copper decreases, the level of ceruloplasmin also decreases, as the amount of plasma ceruloplasmin is dependent upon copper availability.

The term “lowering endogenous copper level” refers to decreasing the copper level in the body of an animal, typically by administration of an agent which binds or complexes copper, from the level existing just before administration of the agent; copper-binding agents include but are not limited to thiomolybdates, of which tetrathiomolybdate is an example. Typically, more than one dose of a copper-binding agent is required to lower the endogenous copper level.

The term “therapeutically effective amount” is a functional term referring to an amount of material needed to make a qualitative or quantitative change in a clinically measured parameter for a particular subject. For example, prior to administration, the subject may exhibit at least one measurable symptom of disease or response to injury (for example, pulmonary congestion and/or difficulty breathing; evidence of hepatitis, or decrease in liver function; evidence or kidney inflammation or decrease in kidney function; etc), which upon administration of a therapeutically effective amount the measurable symptom is found to have changed. A therapeutically relevant effect relieves to some extent one or more symptoms of a disease or condition or returns to normal either partially or completely one or more physiological or biochemical parameters associated with or causative of the disease.

In particular, the term refers to an amount of an agent that binds or complexes copper such as thiomolybdate which amount is effective to treat cardiac damage caused by administration of pharmaceutical agents to a patient suffering from such a disease or response to injury. Treatment includes but is not limited to preventing the onset or shortening the course or severity of or reversing the effects of cardiac damage caused by administration of pharmaceutical agents; thus, a therapeutically effective amount includes a prophylactically effective amount. In some embodiments, such effects are achieved while exhibiting negligible or manageable adverse side effects on normal, healthy tissues of the patient. Thus, the “therapeutically effective amount” can vary from patient to patient, depending upon a number of factors, including but not limited to the type of disease, the extent of the disease, and the size of the patient.

The term “biologically effective amount” is a functional term referring to an amount of material needed to make a qualitative or quantitative change in a biological activity of a particular subject; such activities include but are not limited to enzyme activities, production of antigen, and clearance of analyte from serum.

In particular, the term refers to an amount of an agent that binds or complexes copper such as thiomolybdate which amount is effective to decrease the level of endogenous copper levels upon administration to a patient.

The term “therapeutically effective time” refers to the period of time during which a therapeutically effective amount of a therapeutic agent or biologically active agent is administered sufficient to prevent the onset or to shorten the course or severity of or to reverse the effects of a disease. In particular, it is the period of time sufficient to both reduce the endogenous copper level to a target level and/or to maintain the target copper level to prevent the onset or to shorten the course or severity of or to reverse the effects of cardiac damage caused by administration of pharmaceutical agents.

The term “thiomolybdate” refers to molecules comprising molybdenum and sulfur, and include but are not limited to species such as [MoS₄]²⁻ and [MoO₂S2]²⁻. These molecules can act as bidentate ligands, and can complex copper. Examples of thiomolybdates include but are not limited to tetrathiomolybdate, trithiomolybdate, dithiomolybdate, and monothiomolybdate. Other examples include complex thiomolybdates, which include but are not limited to a zinc or an iron between two thiomolybdate groups, and which contain thiomolybdate capable of binding or complexing copper. In exemplary complex thiomolybdates, the molecule may have more than four thio groups related to more than one molybdenum.

The term “tetrathiomolybdate” (TM) refers to a compound made up of molybdenum atom surrounded by four sulfur groups, [MoS₄]²⁻.

The terms “bind,” “complex,” and “chelate” and any grammatical equivalents (such as “binds,” “binding,” etc.) refer to any type of chemical or molecular interactions of copper with thiomolybdate which effectively sequester the copper with the thiomolybdate; when the copper is endogenous to a patient and thiomolybdate is administered to the patient, the terms refer to any type of chemical interactions of copper with thiomolybdate which effectively sequester the copper with the thiomolybdate rendering the copper unavailable and ultimately removing it from the patient's body.

A therapeutically effective amount of a range of values includes all values in this range. Thus, for example, a therapeutically effective amount of “between about 20 mg and about 200 mg includes all values in this range, and thus includes amounts of about 25 mg, about 30 mg, about 40 mg, about 50 mg, about 60 mg, about 70 mg, about 75 mg, about 80 mg, about 90 mg, about 100 mg, about 1100 mg, about 120 mg, about 125 mg, about 130 mg, about 140 mg, about 150 mg, about 160 mg, about 170 mg, about 180 mg, about 190 mg, or about 195 mg.

The terms “pharmaceutically acceptable,” “physiologically tolerable,” and grammatical variations thereof, as they refer to compositions, carriers, diluents and reagents, are used interchangeably and represent that the materials are capable of administration to or upon a subject or patient, preferably a mammal, most preferably a human, and that the materials do not substantially produce, for example, adverse or allergic reactions when administered to a subject or patient, or can be administered without the production of undesirable physiological effects such as nausea, dizziness, gastric upset, toxicity and the like. As used herein, “pharmaceutically acceptable carrier” includes any and all solvents, dispersion media, coatings, antibacterial and antifungal agents, isotonic and absorption delaying agents and the like. The use of such media and agents for pharmaceutical active substances is well known in the art. Except insofar as any conventional media or agent is incompatible with the active ingredient, its use in the therapeutic compositions is contemplated. Supplementary active ingredients can also be incorporated into the compositions.

The term “aqueous component” refers to the component of a composition that contains water (or is soluble in water). Where water is used, it may or may not contain salt(s) and may or may not be buffered. Thus, a variety of such components are contemplated including, but not limited to, distilled water, deionized water, normal saline, and phosphate buffered saline.

The term “carrier” refers to a diluent, adjuvant, excipient, or vehicle with which a compound is administered. Such pharmaceutical carriers can be sterile liquids, such as water and oils, including those of petroleum, animal, vegetable, or synthetic origin, such as peanut oil, soybean oil, mineral oil, sesame oil and the like. Water or aqueous solution saline solutions and aqueous dextrose and glycerol solutions are preferably employed as carriers, particularly for injectable solutions.

The term “patient” refers to any animal (for example, warm blooded mammal) comprising humans and non-human animals, where non-human animals include but are not limited to non-human primates, rodents, farm animals (for example, cattle, horses, pigs, goats, and sheep), pets (for example, dogs, cats, ferrets, and rodents) and the like, that is to be the recipient of a particular treatment. The terms “patient” and “subject” are used interchangeably. The term “individual” refers to any animal as described above who may or may not be a patient. A patient “having” a disease or condition is a patient “suffering” the disease or condition, and is “in need” of treatment of the disease or condition.

The term “test compound” refers to any chemical entity, pharmaceutical, drug, and the like that can be used to treat or prevent a disease, illness, sickness, or disorder of bodily function. Test compounds comprise both known and potential therapeutic compounds. A test compound can be determined to be therapeutic by using the screening methods of the present invention. A “known therapeutic compound” refers to a therapeutic compound that has been shown (for example, through animal trials or prior experience with administration to humans) to be effective in such treatment or prevention.

The terms “purify” or “to purify” refer to the removal of contaminants from a sample. The term “purified” refers to molecules, such as nucleic or amino acid sequences, that are removed from their natural environment, isolated or separated. An “isolated nucleic acid sequence” is therefore a purified nucleic acid sequence. “Substantially purified” molecules are at least 60% free, preferably at least 75% free, and more preferably at least 90% free from other components with which they are naturally associated. As used herein, the term “purified” or “to purify” also refers to the removal of contaminants from a sample. The removal of contaminating proteins results in an increase in the percent of polypeptide of interest in the sample. In another example, recombinant polypeptides are expressed in plant, bacterial, yeast, or mammalian host cells and the polypeptides are purified by the removal of host cell proteins; the percent of recombinant polypeptides is thereby increased in the sample.

The term “medical devices” includes any material or device that is used on, in, or through a patient's body in the course of medical treatment (for example, for a disease or injury). Medical devices include, but are not limited to, such items as syringes, catheters, intravenous administration assemblies including pumps and monitors, blood sampling equipment, nebulizers, small particle aerosol generators, inhalers with a propellant, and the like.

As used herein, the term “sample” is used in its broadest sense. In one sense, it is meant to include a specimen or culture obtained from any source, as well as biological and environmental samples. Biological samples may be obtained from an animal, including a human; a particular biological sample may be a fluid (for example, blood, plasma and serum), a solid (for example, stool), or a tissue; other biological samples may be obtained from other biological sources, such as food, and may be a liquid food (for example, milk), or a solid food (for example, vegetables). Environmental samples include environmental material such as surface matter, soil, water, crystals, and industrial samples. These examples are not to be construed as limiting the sample types applicable to the present invention.

### DETAILED DESCRIPTION OF THE INVENTION

The present invention relates generally to the field of prophylaxis and therapy for cardiac fibrosis. In particular, the present invention is related to agents that can bind or complex copper, and to the use of these agents in the prevention and treatment of cardiac damage caused by chemotherapeutic and pharmaceutical agents.

The present disclosure provides examples of treatment of myocardial fibrosis, such as that resulting from cumulative anthracycline treatment and dilated cardiac myopathy patients (e.g., with confirmed myocardial fibrosis), with a copper chelator, such as tetrathiomolybdate.

I. Agents that Bind or Complex Copper

### A. Thiomolybdates

The present invention provides methods to prevent and/or treat cardiac damage caused by chemotherapeutic agents by administering a therapeutically effective amount of at least one copper binding or complexing agent that includes but is not limited to a thiomolybdate, to a patient in need thereof. Thiomolybdates are molecules comprising molybdenum and sulfur, and include but are not limited to species such as [MoS₄]²⁻ and [MoO₂S2]²⁻. These molecules can act as bidentate ligands, and can complex copper. Examples of thiomolybdates include but are not limited to tetrathiomolybdate, trithiomolybdate, dithiomolybdate, and monothiomolybdate. Other examples include complex thiomolybdates, that include but are not limited to a zinc or an iron between two thiomolybdate groups, and that contain thiomolybdate capable of binding or complexing copper. In exemplary complex thiomolybdates, the molecule may have more than four thio groups related to more than one molybdenum. In the following description, it is understood that tetrathiomolybdate is simply an embodiment of the use of a copper binding or complexing thiomolybdates. It is also understood that any thiomolybdate may be utilized as one or several of different salts, such as those described for TM below.

Tetrathiomolybdate (TM) is a compound made up of a molybdenum atom surrounded by four sulfur molecules. Various salts of TM are available; salts of TM include but are not limited inorganic cations such as ammonium, zinc, and iron ions, and organic cations such as tetraethyl, tetrapropyl and choline ions. Different salts have differing properties of solubility in water and ingestible solvents (such as alcohol), stability upon storage alone or in formulations, bioavailability to a patient, and toxicity to a patient. Thus, depending upon the use and formulation, any particular salt is selected to maximize solubility in water (or a solvent miscible with water and which can be tolerated by a patient, such as alcohol), to maximize stability upon storage, as for example as the compound or as part of a formulation, to minimize toxicity to a patient, and to maximize bioavailability after administration to a patient.

In some embodiments, the salt of TM is an ammonium salt. TM as the ammonium salt can be purchased from Aldrich Chemical Company (catalog number W 180-0; Milwaukee, Wis.; available in one kilogram bulk lots) as a black powder that is moderately water soluble, yielding a bright red solution; these preparations are also certified pure for human use. The ammonium salt of TM has one undesirable property, that of mild air instability. Thus, the bulk drug should be stored in the absence of oxygen, or the oxygen will gradually exchange with the sulfur, rendering the drug ineffective over time. The bulk drug is therefore stored under argon. Stability assays developed by the inventors indicate that this drug is stable for several years under argon (G J Brewer et al., Arch. Neurol., 48(1):42-47 [1991]). Capsules can be filled by hand, and the drug is stable in capsules for several months at room temperature.

Alternatively, TM, which is generally synthesized as the ammonium salt, may be more stable under air as a different salt. Thus, other salts have been prepared and evaluated for solubility, stability and anticopper activity. In other embodiments, the salt tetrapropyl tetrathiomolybdate (TPTM) has met all desired properties. In other embodiments, the salt choline TM has suitable desirable properties. In yet other embodiments, the salt tetraethyl TM has suitable properties. These exemplary salts of TM have suitable solubility in water, and behave similarly to ammonium salt of TM in in vitro copper complexing studies.

Other pharmaceutically acceptable salts include but are not limited to include salts formed with inorganic acids such as, for example, hydrochloric or phosphoric acids, or such organic acids as acetic, oxalic, tartaric, mandelic, and the like. Salts can also be derived from inorganic bases such as, for example, sodium, potassium, ammonium, calcium, or ferric hydroxides, and such organic bases as isopropylamine, trimethylamine, histidine, procaine and the like.

Although it is not necessary to understand the underlying mechanism to practice the invention, and the invention is not intended to be so limited, it is believed that TM acts by forming a tripartite complex with copper and protein (Mills et al., J. Inorg. Biochem., 14:189 [1981]; Mills et al, J. Inorg. Biochem., 14:163 [1981]; and Bremner et al., J. Inorg. Biochem., 16:109 [1982]). It is further believed that TM has two mechanisms of action. Given with meals, it complexes both copper in food and endogenously secreted copper with itself and food protein, and prevents the absorption of copper. Patients can be put into an immediate negative copper balance with TM by administering it with meals. Given between meals, the TM is absorbed into the bloodstream, and complexes serum copper with itself and albumin, rapidly rendering the copper unavailable for cellular uptake. Since free copper in organs is in equilibrium with free copper in blood, free copper in the organs will quickly be reduced to very low levels if the blood copper is bound. This complex is cleared through the kidney and the liver. No matter what its mechanism of action is, TM is a potent and rapidly acting anticopper agent. It is also contemplated that other thiomolybdates complex copper through similar though not necessarily identical mechanisms. In some hypotheses, some thiomolybdates directly bind copper. In yet other hypotheses, a tripartite copper-thiomolybdate-protein complex is degraded in the body, but the thiomolybdate still results in lowering endogenous copper levels. For example, trithiomolybdate, dithiomolybdate, and monothiomolybdate compounds, like tetrathiomolybdate, are believed to form a tripartite complex with copper and protein that renders the copper unavailable, and eventually leads to clearance of the copper-complex.

The only known toxicity of TM discovered in animal studies is through its anticopper effects. Animals given TM in sufficient quantity to produce severe copper deficiency suffer from a variety of copper deficiency-related problems, including anemia (Mills et al., J. Inorg. Biochem., 14:163 [1981]; and Bremner et al., J. Inorg. Biochem., 16:109 [1982]). However, none of these occur if the animal is copper supplemented (Mills et al., J. Inorg. Biochem., 14:189 [1981]), or maintained at a moderate copper level. Tetrathiomolybdate (TM) is a drug that controls over copper toxicity and prevents the neurological worsening that occurs 50% of the time during initial treatment with a commonly used drug for Wilson's disease, penicillamine (Brewer et al., Arch. Neurol., 48(1):42-47 [1991]; Brewer et al., Arch. Neurol., 51(6):545-554 [1994]; and Brewer et al., Arch. Neurol., 53:1017-1025 [1996]). TM thus fills a very important niche in the initial treatment of Wilson's disease. The Wilson's disease work has provided extensive experience with TM therapy in the human, and provides documentation of TM's extremely low level of toxicity in humans.

In the studies of treating human patients with Wilson's disease studies, one side effect occasionally observed is a reversible anemia, due to TM's anticopper effects. Given in too high a dose, TM renders the bone marrow severely or totally copper deficient. Since copper is required for erythropoiesis, an anemia develops. That anemia is rapidly reversible by simply stopping TM. In the Wilson's disease studies, this over-treatment effect of TM has been diminished by simply reducing the dose to 60 mg per day from the standard 120 mg per day. A second side effect seen during treatment with TM of Wilson's disease, but not in treatment of cancer, is a mild increase in serum transaminase levels (Wilson's patients already have liver disease). This mild increase is diminished or removed by reducing the dose of TM. In humans without Wilson's disease, a level of mild copper deficiency at a pre-anemia state can be established simply by carefully monitoring ceruloplasmin (Cp) levels during TM therapy. The level of ceruloplasmin is reduced to and maintained at a targeted level; in some embodiments, this targeted level is between about 5 and 15 mg/dl.

TM is eventually metabolized to elemental molybdenum (Mo), so the potential toxicity of Mo has to be considered. However, it turns out that Mo is quite innocuous at the levels produced from breakdown of TM used at the therapeutic regimes described herein. In one example, up to 50 mg of Mo/day is administered for two weeks, then no more than about 25 mg/day is administered for maintenance. High doses of 350 to 1400 mg/day of Mo were previously used for 4-11 months in patients with Wilson's disease, without toxicity (Bickel et al., Quart. J. Med., 50:527 [1957]). Thus, because about 37% of TM by weight is Mo, the dose range of 25-50 mg/day poses no predictable problems, and is entirely safe.

### B. Monitoring Copper Levels

The mechanism of action of TM is to lower systemic or endogenous copper levels. Copper status is evaluated by measuring the level of ceruloplasmin, a copper-containing serum protein secreted by the liver, as the amount of ceruloplasmin is dependent upon copper availability. Measuring total serum copper is not a good indicator for evaluating copper status, because the TM complex with copper accumulates in the blood before it is cleared from the body, thus elevating serum copper in spite of reduced copper availability. Thus, the serum ceruloplasmin, which is directly dependent upon liver copper status, is an accurate indicator of copper status or availability in preferred embodiments.

### II. Combination Therapy

In the present invention, it is initially contemplated that a method to treat cardiac damage induced by chemotherapeutic agents comprises administration of at least one copper binding or complexing agent, which include but are not limited to thiomolybdates of which TM is an example; in this method, treatment is accomplished by administering a single copper binding or complexing agent. It is also contemplated that a combination of more than one copper binding or complexing agent may be administered to a patient; the different agents are chosen from different thiomolybdates, different salts of different thiomolybdates, other copper binding or complexing agents, or any combination thereof. Thus, in some embodiments, the agents comprise a combination of at least two different thiomolybdates, such as a tetrathiomolybdate and a trithiomolybdate; in other embodiments, the agents comprise a combination of at least one thiomolybdate and at least one other copper binding or complexing agents. In yet other embodiments, the agents comprise a combination of at least two different salts of a single thiomolybdate, such as a tetraethyl- and a tetrapropyl-tetrathiomolybdate; in yet other embodiments, the agents comprise a combination of at least two different thiomolybdates, of which at least one thiomolybdate comprises at least two different salts; in yet other embodiments, the agents comprise a combination of at least one thiomolybdate, which comprises a combination of at least two different salts, and at least one other copper binding or complexing agent.

Moreover, it is also contemplated that the methods of the present invention may be combined with other methods generally employed in the treatment of the particular disease or disorder that the patient exhibits. This is particularly true for treatment of diseases for which decreasing copper levels ameliorates does not eradicate the disease; in those cases, it may be advantageous to use additional compounds which eradicate the disease. In other cases, it may be useful to administer drugs in addition to TM in order to obtain additive or synergistic effects. In some examples, in connection with fibrosis (e.g., cardiac fibrosis), the methods of the present invention include classical or new approaches in treating and/or preventing fibrosis. Thus, in some embodiments, the present invention provides a method of treating and/or preventing fibrosis comprising administering at least one copper binding or complexing agent as described above to a patient in need thereof, and administering at least one other known or discovered anti-fibrotic drugs; anti-fibrotic drugs include but are not limited to antibodies or antisense agents directed to specific cytokines or to their receptors, as well as to other molecules which enhance fibrosis. In these embodiments, it is contemplated that the administration of other drugs are not known to be detrimental in themselves, and that administration of other drugs do not substantially counteract the effectiveness of the endogenous copper lowering therapy by administering copper binding or complexing agents. By substantially counteracting the effectiveness of the endogenous copper lowering therapy, it is meant that the combined therapy lowers endogenous copper sufficiently to observe an amelioration of at least one symptom of a disease or condition. In the embodiments in which at least one additional drug is administered in combination with the administration of a copper binding or complexing agent, there is no requirement for the combined results to be additive of the effects observed when each treatment is conducted separately, although this is evidently desirable, and there is no particular requirement for the combined treatment to exhibit synergistic effects, although this is certainly possible and advantageous. It is also contemplated that the administration of the different agents or drugs occurs simultaneously, as for example administering the combination of agents and drugs at the same times, and/or at different times during the course of therapy; any combination of administration is contemplated.

### III. Pharmaceutical Compositions and Kits

Pharmaceutical compositions of the present invention will generally comprise an effective amount of an agent for use in the present invention, such as copper binding or complexing thiomolybdates, of which tetrathiomolybdate is an example, dissolved or dispersed in a pharmaceutically acceptable carrier or aqueous medium.

### A. Oral Formulations

In some embodiments of the present invention, TM is administered orally. Oral administration is effected by a number of means, such as by feeding tubes for administration into the gastrointestinal track, and preferably the duodenum, or by tablets or powders or solutions for administration by mouth. A feeding tube may be preferred for an acute disease, whereas administration by mouth may be preferred for chronic diseases and/or for maintenance, once an appropriate level of copper has been attained. Oral pharmaceutical formulations include but are not limited to tablets or other solids, time or slow release capsules, liposomal forms and the like. Other pharmaceutical formulations may also be used, dependent on the condition to be treated.

As described in detail herein, it is contemplated that certain benefits will result from the manipulation of the agents for use in the present invention, such as copper binding or complexing thiomolybdates, to provide them with a longer in vivo half-life. Slow release formulations are generally designed to give a constant drug level over an extended period. Increasing the half-life of a drug, such as agents for use in the present invention, such as copper binding or complexing thiomolybdates, is intended to result in high plasma levels of TM upon administration, which levels are maintained for a longer time, but which levels generally decay depending on the pharmacokinetics of the construct. Slow release formulations of the instant compositions and combinations thereof are contemplated for some uses in the present invention.

Appropriate solutions of the agents for use in the present invention, such as copper binding or complexing thiomolybdates, of which tetrathiomolybdate is an example, pharmaceutical forms suitable for administration, compositions comprising the agents, formulations with the agents, and carriers may be similar to those described below.

### B. Parenteral Formulations

In addition to the compounds formulated for parenteral administration, the agents for use in the present invention, such as copper binding or complexing thiomolybdates, may be formulated for parenteral administration, e.g., formulated for injection via the intravenous, intramuscular, sub-cutaneous or other such routes, including direct instillation into a disease site. The preparation of an aqueous composition that contains one or more agents for use in the present invention, such as copper binding or complexing thiomolybdates, as an active ingredient will be known to those of skill in the art in light of the present disclosure. Typically, such compositions can be prepared as injectables, either as liquid solutions or suspensions; solid forms suitable for using to prepare solutions or suspensions upon the addition of a liquid prior to injection can also be prepared; and the preparations can also be emulsified.

Solutions of the active compounds as freebase or pharmacologically acceptable salts can be prepared in water suitably mixed with a surfactant, such as hydroxypropylcellulose. Dispersions can also be prepared in glycerol, liquid polyethylene glycols, and mixtures thereof and in oils. Under ordinary conditions of storage and use, these preparations contain a preservative to prevent the growth of microorganisms.

The pharmaceutical forms suitable for injectable use include sterile aqueous solutions or dispersions; formulations including sesame oil, peanut oil or aqueous propylene glycol; and sterile powders for the extemporaneous preparation of sterile injectable solutions or dispersions. In all cases the form should be sterile and should be fluid to the extent that easy flow through a syringe exists. It should be stable under the conditions of manufacture and storage and must be preserved against the contaminating action of microorganisms, such as bacteria and fungi.

Compositions comprising the agents for use in the present invention, such as copper binding or complexing thiomolybdates, can be formulated into a composition in a neutral or salt form. Pharmaceutically acceptable salts have been described above.

The carrier can also be a solvent or dispersion medium containing, for example, water, ethanol, polyol (for example, glycerol, propylene glycol, and liquid polyethylene glycol, and the like), suitable mixtures thereof, and vegetable oils. Proper fluidity can be maintained, for example, by the use of a coating, such as lecithin, by the maintenance of the required particle size in the case of dispersion and by the use of surfactants. Prevention of the contamination with microorganisms can be obtained by various antibacterial and antifungal agents, for example, parabens, chlorobutanol, phenol, sorbic acid, thimerosal, and the like. In many cases, it may be desirable to include isotonic agents, for example, sugars or sodium chloride. Prolonged absorption of the injectable compositions can be brought about by the use in the compositions of agents delaying absorption, for example, aluminum monostearate and gelatin.

Sterile injectable solutions are prepared by incorporating the active compounds in the required amount in the appropriate solvent followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum-drying and freeze-drying techniques which yield a powder of the active ingredient plus any additional desired ingredient from a previously sterile-filtered solution thereof.

Upon formulation, solutions will be administered in a manner compatible with the dosage formulation and in such amount as is therapeutically effective. Formulations are easily administered in a variety of dosage forms, such as the type of injectable solutions described above, but drug release capsules and the like can also be employed.

Suitable pharmaceutical compositions in accordance with the invention will generally include an amount of one or more of the agents for use in the present invention, such as copper binding or complexing thiomolybdates, admixed with an acceptable pharmaceutical diluent or excipient, such as a sterile aqueous solution, to give a range of final concentrations, depending on the intended use. The techniques of preparation are generally well known in the art as exemplified by Remington's Pharmaceutical Sciences, 16th Ed. Mack Publishing Company, 1980, incorporated herein by reference. It should be appreciated that endotoxin contamination should be kept minimally at a safe level, for example, less that 0.5 ng/mg protein. Moreover, for human administration, preparations should meet sterility, pyrogenicity, general safety and purity standards as required by FDA Office of Biological Standards.

The therapeutically effective doses are readily determinable using an animal model, as shown in the studies detailed herein. Experimental animals with induced diseases are frequently used to optimize appropriate therapeutic doses prior to translating to a clinical environment. Such models are known to be very reliable in predicting effective anti-fibrotic strategies. One can use such art-accepted mouse models to determine working ranges of agents for use in the present invention, such as copper binding or complexing thiomolybdates, that give beneficial effects with minimal toxicity.

### C. Therapeutic Kits

The present invention also provides therapeutic kits comprising agents for use in the present invention which bind or complex copper, such as thiomolybdates, and of which tetrathiomolybdate is an example, as described herein. Such kits generally comprise, in suitable container means, a pharmaceutically acceptable formulation of at least one agent for use in the present invention, such as copper binding or complexing thiomolybdates, in accordance with the invention. The kits may also comprise other pharmaceutically acceptable formulations, such as any one or more of a range of anti-inflammatory and/or anti-fibrotic drugs.

The kits may have a single container means comprising an agent that binds or complexes copper, such as a thiomolybdate, with or without any additional components, or they may have distinct container means for each desired agent. In some embodiments, kits of the present invention comprise an agent for use in the present invention, such as copper binding or complexing thiomolybdates, packaged in a kit for use in combination with the co-administration of a second agent, such as an anti-inflammatory or anti-fibrotic agent as described above. In such kits, the components may be pre-complexed, either in a molar equivalent combination, or with one component in excess of the other; or each of the components of the kit may be maintained separately within distinct containers prior to administration to a patient.

When the components of the kit are provided in one or more liquid solutions, the liquid solution is generally an aqueous solution, with a sterile aqueous solution being particularly preferred. However, the components of the kit may be provided as dried powder(s). When reagents or components are provided as a dry powder, the powder can be reconstituted by the addition of a suitable solvent. It is envisioned that the solvent may also be provided in another container means. One of the components of the kit may be provided in capsules for oral administration.

The container means of the kit will generally include at least one vial, test tube, flask, bottle, syringe or other container means, into which an agent for use in the present invention, such as copper binding or complexing thiomolybdates, and any other desired agent, may be placed and, preferably, suitably aliquoted. Where additional components are included, the kit will also generally include a second vial or other container into which these additional components are placed, enabling the administration of separated designed doses. The kits may also comprise a second and/or third container means for containing a sterile, pharmaceutically acceptable buffer or other diluent.

The kits may also comprise a means by which to administer an agent for use in the present invention, such as copper binding or complexing thiomolybdates, to an animal or human patient, e.g., one or more needles or syringes, or even an eye dropper, pipette, or other such like apparatus, from which the formulation may be injected into the animal or applied to a feeding tube or ingested orally. The kits of the present invention will also typically include a means for containing the vials, or such like, and other component, in close confinement for commercial sale, such as, e.g., injection or blow-molded plastic containers into which the desired vials and other apparatus are placed and retained.

For human use, in preferred embodiments, the kits further comprise appropriate instructions and labels (e.g., as required by the FDA) for use of copper binding or complexing agents as described herein.

### IV. Treatment of Cardiac Diseases

As described above, in some embodiments, the present invention provides methods of treating and/or preventing cardiac disease (e.g., dilated cardiomyopathy or cardiac disease caused by chemotherapeutic or pharmaceutical agents.

Dilated cardiomyopathy or DCM, is a severe condition generally associated with poor outcomes. DCM may be caused by or associated with myocardial fibrosis confirmed either by biopsy or more recently, by non-invasive imaging means such as delayed hyperenhancement magnetic resonance imaging. Such technique has been shown to be highly useful for stratifying DCM patients based upon their potential to recover or progress. The cardiac muscle, though involuntary, is striated like skeletal muscle. Striated muscle is the muscle tissue named so because of its striated appearance of the sarcomere due to the alignment of dark bands (myosin filament region) and light bands (Z-line region at each end of sarcomere) caused by the arrangement of actin filaments and myosin filaments in the myofibrils. Muscle cell is an elongate fibrous cell, containing numerous myofibrils and mitochondria in its cytoplasm (sarcoplasm), and is enclosed within the cell membrane called sarcolemma (sarcoplasmic membrane).

A myofibril in the sarcoplasm consists of sarcomeres which are the assemblies of actin filaments and myosin filaments. The thinner transverse line of Z-line exists in between sarcomeres. T-tubule is located close to the Z-line, which instantly propagates the electrical excitation transmitted over the surface of sarcolemma into a myofibril, the interior of muscle cell. The electrical excitation propagated into muscle cell is then led to sarcomere via T-tubule, thereby initiated a contraction.

Cardiac muscle made up with the reticulum of muscle fibers (cardiac muscle cells) that spirally surrounds the heart, delivers blood to the whole body by repeated autonomous contractions. When the heart is overloaded for a long period, cardiac muscle tissue enlarges itself not by cell division but increasing length and width of each cardiac muscle cell. That is called myocardial hypertrophy. When the weight of the heart exceeds 500 g, insufficient supply of blood to cardiac muscle cells occurs because the coronary artery or the like which supply oxygen and nutrition to cardiac muscle cells are undevelopable in compensation for the hypertrophy thereof.

Cardiomyopathy is a disease resulting from abnormality of myocardium. Heart being a life-sustaining organ, and yet non-regenerative, cardiomyopathy is a severe progressive degenerative disease thereof. Cardiomyopathy is classified into a primary cardiomyopathy such as dilated (congestive) and hypertrophic cardiomyopathy; and a secondary cardiomyopathy such as infectious cardiomyopathy. The primary cardiomyopathy is categorized into hypertrophic cardiomyopathy caused by hypertrophy of cardiac muscle and dilated cardiomyopathy caused by other factors.

The present invention thus provides methods of treating cardiomyopathy such as DCM (e.g., resulting from administration of chemotherapeutic or pharmaceutical agents) by administering copper lowering compounds (e.g., those described herein).

Experiments conducted during the course of development of the present invention compared the efficacy of zinc with tetrathiomolybdate on the doxorubicin model of heart damage, and contemplates that when copper availability is lowered to an equivalent extent, the two drugs show equivalent efficacy. However, it was found that at similar levels of copper lowering at the time of doxorubicin exposure, tetrathiomolybdate is much more effective than zinc at protecting against cardiac damage. The present invention is not limited to a particular mechanism. Indeed, an understanding of the mechanism is not necessary to practice the present invention. Nonetheless, it is contemplated that the differences between tetrathiomolybdate and zinc may be due to tetrathiomolybdate's mechanism of action in which it binds copper already in the body, while zinc does not.

The present invention is not limited to a particular mechanism. Indeed, an understanding of the mechanism is not necessary to practice the present invention. Nonetheless, in other experiments it was shown that tetrathiomolybdate's mechanism of action in protecting against doxorubicin toxicity is due to its anticopper effects, because copper supplementation eliminated the protective effect of tetrathiomolybdate.

In still other experiments, the protective effect on the heart against doxorubicin toxicity of tetrathiomolybdate is compared with another anticopper drug, trientine, in the same animal model. Again, it was found that tetrathiomolybdate was much more effective than trientine in protecting the heart. The present invention is not limited to a particular mechanism. Indeed, an understanding of the mechanism is not necessary to practice the present invention. Nonetheless, it is contemplated that this difference is due to the differences in mechanism of action in binding free copper in the body.

It is contemplated that the methods of the present invention are widely or entirely applicable to the treatment of cardiac disease and damage. However, the particular type of disease may be relevant to the use of the methods of the present invention in combination with secondary therapeutic agents, as described above.

There are realistic objectives that may be used as a guideline in connection with pre-clinical testing before proceeding to clinical treatment. However, this is generally more a matter of cost-effectiveness than overall usefulness, and is a means for selecting the most advantageous compounds and doses. In regard to their basic utility, any composition or combination comprising a copper binding or complexing agent such as thiomolybdate that results in any consistent anti-inflammatory and/or anti-fibrotic effects defines a useful compound. Even in those circumstances where the anti-inflammatory and/or anti-fibrotic effects are towards the low end of the range, it may be that the therapy of the present invention is as or even more effective than other known therapies in the context of particular types of cardiac damage, and especially where other factors (such as desirable or undesirable side effects, or quality of life) may be important. Even if it becomes evident to the clinician that particular diseases cannot be effectively treated in the intermediate or long term, it does not negate the utility of the therapy of the present invention, particularly where it is about as effective as other known strategies, or where it is effective after other conventional therapies have failed. It is not predicted that resistance to therapy of the present invention can develop.

In the present invention, an agent that binds or complexes copper such as a thiomolybdate is administered in a therapeutically effective amount to a patient suffering from cardiac disease or damage. The term “therapeutically effective amount” is a functional term referring to an amount of material needed to make a qualitative or quantitative change in a clinically measured parameter for a particular subject. For example, prior to administration, the subject may exhibit measurable symptoms of disease (for example, decreased heart function or other symptoms of cardiac disease or damage, etc), which upon administration of a therapeutically effective amount the measurable symptom is found to change over time. A therapeutically relevant effect relieves to some extent one or more symptoms of a disease or condition or returns to normal either partially or completely one or more physiological or biochemical parameters associated with or causative of the disease.

In particular, the term refers to an amount of an agent that binds or complexes copper such as thiomolybdate effective to treat cardiac disease or damage upon administration to a patient suffering from such a disease. Treatment includes but is not limited to preventing the onset or shortening the course or severity of or reversing the effects of disease; thus, a therapeutically effective amount includes a prophylactically effective amount. Such effects are achieved while exhibiting negligible or manageable adverse side effects on normal, healthy tissues of the patient. Thus, the “therapeutically effective amount” can vary from patient to patient, depending upon a number of factors, including but not limited to the type of disease, the extent of the disease, and the size of the patient.

An objective of the therapeutic regimes of the present invention is to reduce the endogenous copper level to a target level, and then to maintain that level for a period of time sufficient to prevent the onset or to shorten the course or severity of or to reverse the effects of cardiac disease or damage. The period of time sufficient to both reduce endogenous copper level and to maintain it to prevent the onset or to shorten the course or severity of or to reverse the effects of inflammatory and/or fibrotic disease is referred to as a “therapeutically effective time”. As described earlier, the level of endogenous copper can be monitored by measuring blood ceruloplasmin (Cp) levels. In some embodiments, the levels of blood ceruloplasmin decrease by 10%; in other embodiments, these levels decrease by about 25%; in yet other embodiments, these levels decrease by about 50%; in still other embodiments, these levels decrease by about 90%. Alternatively, ceruloplasmin levels decrease to between about 5 to 15 mg/dl. The time period in which to reduce endogenous copper levels will vary, depending upon the disease and the patient's general health and condition; typically, this time depends upon the amount of copper-binding agent per dose, and frequency of dose administration per treatment period. Generally, for acute diseases, it is desirable to decrease endogenous copper levels as rapidly as possible; this is because patients are at risk of dying quickly, and it is therefore desirable to initiate quick intervention. Under these circumstances, it is preferable to use initially a much higher loading dose of a copper-binding or complexing agent than might be used for a chronic disease or condition. For both acute and chronic diseases, initial doses of copper binding or complexing agents might be higher and administered more frequently in order to fairly rapidly decrease endogenous copper to the target levels; these doses are referred to as induction doses. Subsequent doses of copper binding or complexing agents to maintain endogenous copper at the target level may be lower, and administered less frequently; these doses are referred to as maintenance doses.

In designing appropriate doses of the agents that bind or complex copper such as thiomolybdate and combinations therewith, and/or that effectively lower endogenous copper, one may readily extrapolate from animal studies, as for example as described further below, in order to arrive at appropriate doses for clinical administration. To achieve this conversion, one would account for the mass of the agents administered per unit mass of the experimental animal, and yet account for the differences in the body surface area between the experimental animal and the human patient. All such calculations are well-known and routine to those of ordinary skill in the art. Accordingly, using the information provided herein, it is contemplated that useful daily doses of the agents that bind or complex copper such as thiomolybdate, and/or that effectively lower endogenous copper, for use in human administration would be between about 20 mg and about 200 mg per patient per day. Notwithstanding this stated range, it is contemplated that, given the parameters and guidance described above, further variations in the active or optimal ranges are encompassed within the present invention.

Induction doses contemplated are generally about 180 mg per day. Daily maintenance doses contemplated are generally between about 20 mg and about 180 mg; between about 25 and about 160 mg; between about 50 and about 150 mg; between about 30 and about 125 mg; between about 40 mg and about 100 mg; between about 35 and about 80 mg; between about 20 and about 65 mg; between about 30 mg and about 50 mg; about 40 mg; or in any particular range using any of the foregoing recited exemplary doses or any value intermediate between the particular stated ranges. Although daily doses in and around about 60 mg to about 120 mg, or in and around about 20 to about 180 mg, are typical, it is contemplated that lower doses may be more appropriate in combination with other agents, or under conditions of maintenance, and that high doses can still be tolerated, particularly given the fact that the agents that bind or complex copper such as a thiomolybdate and/or that effectively lower endogenous copper for use in the invention are not themselves cytotoxic. Even if certain adverse side effects do occur, this should not necessarily result in toxicity that cannot be counteracted by normal homeostatic mechanisms, which is believed to lessen the chances of significant toxicity to healthy tissues.

The values described above can also be expressed in terms of mg/kg of body weight. As described above, the biologically or therapeutically effective amount can vary, depending upon the size of the animal or human patient. However, taking the average weight of a human male as about 70 kg, the biologically or therapeutically effective amount of the agent that binds or complexes copper such as a thiomolybdate for an average human male would be between about 0.3 mg/kg and about 3 mg/kg.

Another objective of the therapeutic regimes of the present invention is generally to produce the maximum anti-fibrotic or cardioprotective effects while keeping the dose below the levels associated with unacceptable toxicity. However, as noted above, in acute diseases or conditions, it may be necessary to administer initial high doses to rapidly decrease endogenous copper levels. In addition to varying the dose itself, the administration regimen can also be adapted to optimize the treatment strategy. A currently preferred maintenance treatment strategy is to administer between about 20 mg and about 200 mg of the agents that bind or complex copper such as a thiomolybdate or combination thereof, or which effectively lower endogenous copper, from about 3 to about 6 or more times per day, approximately half of the doses with meals, and approximately half of the doses between meals. In administering the particular doses themselves, one would preferably provide a pharmaceutically acceptable composition to the patient systemically. Oral administration is generally preferred. An exemplary induction dosage regime is about 40 mg three times daily with meals, and about 60 mg at bedtime. Exemplary maintenance dosage regimes are about 20 to 180 mg total per day, taken approximately proportionately as indicated for the exemplary induction dosage regime, or taken at fewer times per day, for example, with breakfast and with dinner.

### EXAMPLES

The following examples are provided in order to demonstrate and further illustrate certain preferred embodiments and aspects of the present invention and are not to be construed as limiting the scope thereof.

### Example 1

### A. METHODS

Mice and mouse food. All mice were 7-8 weeks old at the start of the various studies, were C57BL/6 purchased from Jackson Laboratory, Bar Harbor, Me., and were housed at 21±2° C. on a 12 hr light/dark cycle in polycarbonate cages containing corn cob bedding. Experimental animals were housed in the University of Michigan Unit for Laboratory Animal Medicine facility and treated in accord with a protocol approved by the University of Michigan Institutional Animal Care and Use Committee. They were fed a mouse diet from Harlan/Teklad, Madison, Wis., to which copper and zinc had not been added by the manufacturer. Each lot was assayed by atomic absorption and found to contain about 0.6 mg of copper and about 6.0 mg of zinc/kg of food. Copper and zinc are then added to the diet such that the final contents were 2 mg copper and 10 mg zinc/kg of food.

Materials. DXR was purchased from Pharmacia and Upjohn Co, Kalamazoo, Mich. Tetrathiomolybdate was synthesized by Dr. Dimitri Coucouvanis in the chemistry department, University of Michigan. Trientine was purchased from Sigma Chemicals.

Zinc vs. TM therapy mouse doxorubicin study. The zinc depot method involved injecting subcutaneously a 20 mg zinc/mL of corn oil suspension to achieve a dose of 110 mg of zinc/kg of mouse weight, weekly. With this method, Cp levels were lowered to target range in 4 weeks. Cp levels were lowered with TM in a second group of mice by using 0.015 mg/ml of TM in the drinking water, and changing the drinking water every day.

20 male mice were divided into four equal groups, 5 to receive depot zinc and then DXR, 5 to receive TM in the drinking water and then DXR, 5 to receive DXR only, and 5 to serve as controls. After Cp levels had been lowered in the first two groups, the first three groups of mice received DXR intraperitoneally at a single dose of 20 mg/kg body weight. Since DXR toxicity inhibits mice from drinking water, after DXR administration, TM mice were treated with a daily gavage of 0.2 mg of TM in 0.1 ml of water. Four days after DXR, all mice were sacrificed, and Cp, troponin I, creatine kinase (CK) MB, and lactic dehydrogenase (LDH) levels were all measured in the plasma according to previously described methods.

Trientine mouse doxorubicin study. The copper chelator trientine was studied and compared to TM to determine the effects, if any, of trientine on DXR induced cardiotoxicity and myocardial fibrosis in mice using doses of trientine by oral gavage to lower the Cp to target range.

TM therapy copper supplementation doxorubicin study. In order to verify that the effect of TM in the DXR study was due to lowering copper, a study of DXR toxicity was done in which a TM treated group was supplemented with excess copper. In this study, 20 mice were divided into four equal groups, 5 to receive 0.015 mg/ml of TM in the drinking water to lower Cp and then receive DXR (TM was given by gavage after DRX), 5 to receive TM in the same dose and manner as the first group but the diet was supplemented with either 30 or 100 ppm copper such that the Cp didn't fall as far as with TM alone, and then they received DXR, 5 to receive DXR only, and 5 served as controls. After Cp was decreased in the first group, DXR in an intraperitoneal dose of 20 mg/kg body weight was given to the first three groups of mice. Four days after DXR the mice were sacrificed and serum Cp, troponin I, and LDH determined.

Assay Methods. Cp was measured by the oxidase enzyme using known methods. Troponin I, LDH, and CK MB were measured by kit methods using known methods.

Statistical Analysis. For the comparison of means analysis of variance (ANOVA) was used. All data in the figures are given as means+the standard error. Significantly different values at p=0.05 or less are marked with asterisks.

### B. RESULTS

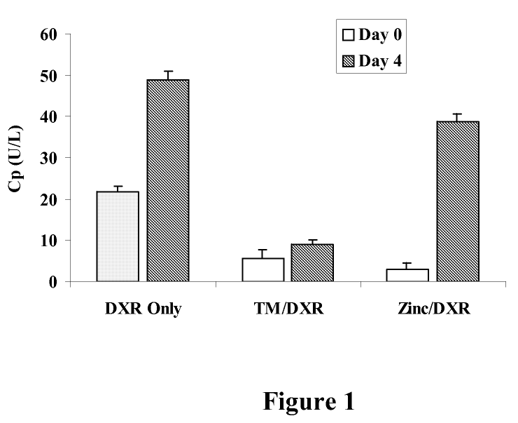

Zinc therapy mouse DXR study. The Cp values of the three groups of mice that received DXR at day 0 (day of DXR administration) and at day 4 (day of sacrifice) are shown in FIG. 1. It is clear that the zinc treated animals and the TM treated animals had equal suppression of Cp at day 0. It can be seen that Cp in DXR only animals had increased at day 4. This is due to Cp being an acute phase reactant, and it increases in response to the inflammation from DXR damage. A similar increase, although not quite as large, is seen at day 4 in the zinc treated animals, but this increase is almost completely prevented in the TM treated animals.

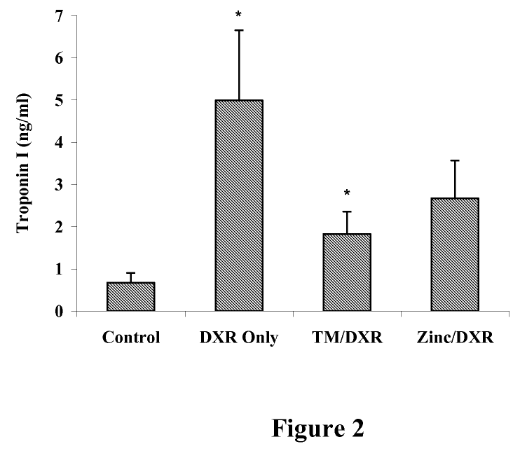

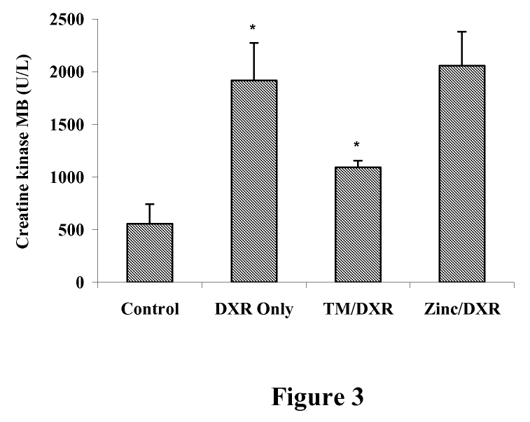

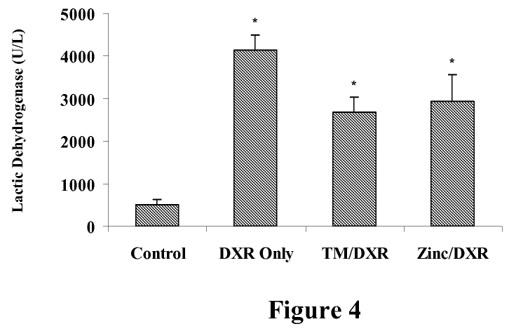

The day 4 troponin I values are shown in FIG. 2. TM had a statistically significant effect on maintaining a lower troponin I (p<0.03). Zinc treated animals had a somewhat lower mean value than controls, but it didn't reach statistical significance (p<0.12). The day 4 CK MB values are shown in FIG. 3. TM therapy resulted in a statistically significantly lower value than DXR controls (p<0.04), but zinc treatment had no effect. The day 4 LDH values are shown in FIG. 4. TM had a statistically significant effect on maintaining lower values than DXR controls (p=0.02). Zinc treatment also had a statistically significant effect (p=0.05).

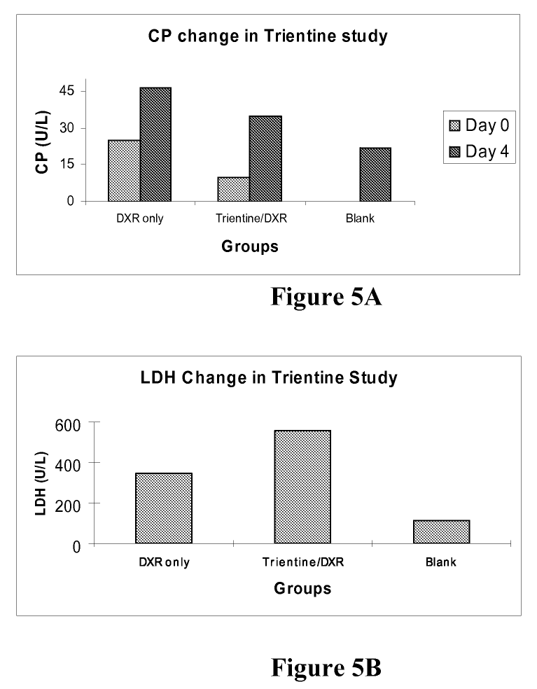

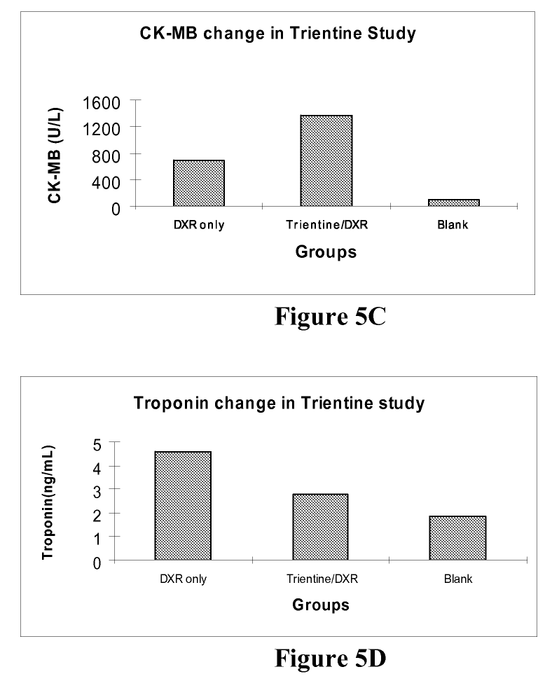

Trientine mouse doxorubicin study. FIG. 5 is a bar graph of the results of a comparative study of trientine in the doxorubicin heart model. Animals were pretreated to lower copper as measured by ceruloplasmin (Cp). The first graph shows that at day 0 (when DXR is given), Cp was down significantly, into the target range with trientine treatment. However, at day 4 (4 days after DXR), the Cp in the trientine group increased into the normal range, in spite of continued trientine therapy by oral gavage. Note that Cp also increased in the DXR only group. These increases in Cp are due to an acute phase response, that is, the inflammation caused by DXR induces some proteins, including Cp, to increase. However, Cp can only increase if copper is available. Thus, the increase in the trientine treatment group means enough free copper is available to allow the increase in Cp. This is in contrast to the control of Cp even 4 days after doxorubicin, by TM (FIG. 1).

FIGS. 5B and 5C show that trientine had no protective effect on LDH and CK-MB respectively. FIG. 5D shows that trientine had a statistically insignificant effect on Troponin 1.

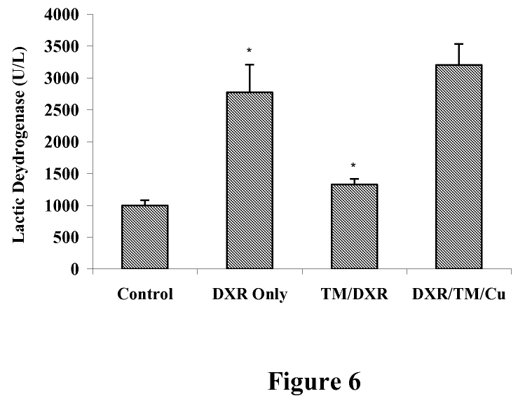

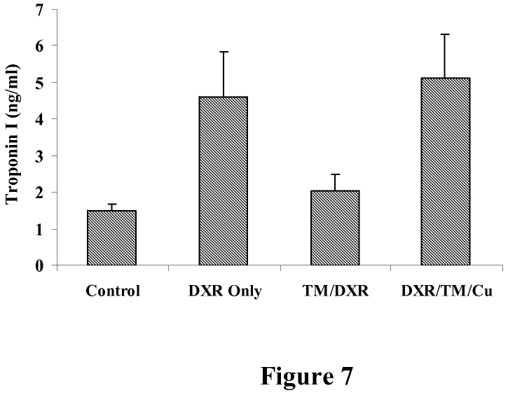

TM therapy copper supplementation DXR study. On day zero, the day of DXR administration, the Cp value of the DXR only group averaged about 25 U/L, the Cp value of the TM group was lowered to about 12 U/L, and the copper supplemented TM group to about 17.5 U/L. The results in the two copper supplemented groups (30 and 100 ppm) were combined since all results in the two groups were similar. Thus copper supplementation partially nullified the effect of TM on Cp suppression. At day 4 after DXR administration the animals were sacrificed and plasma LDH and troponin I measured. FIG. 6 presents the LDH data. Consistent with the results in the previous experiment, the LDH levels of TM/DXR treated animals are statistically significantly lower than in the DXR only animals (p=0.006), and copper supplementation completely eliminated this effect of TM. FIG. 7 presents the troponin I data. The mean troponin I levels of TM/DXR treated animals are less than half of the DXR only animals, although this escaped statistical significance (p=0.15) because of high variances. Copper supplementation completely eliminated the TM effect, and copper supplemented animals had values equivalent to DXR only controls.

As shown in the last experiment presented, with copper supplementation, the effect of TM on protecting against DXR cardiac damage is due to its copper lowering effects. Copper supplementation completely eliminated the protective effect of TM on the heart as reflected in LDH (FIG. 6) and troponin I (FIG. 7) plasma levels. The present invention is not limited to a particular mechanism. Indeed, an understanding of the mechanism is not necessary to practice the present invention. Nonetheless, these findings make it unlikely that some effect of TM other than its effect on copper availability account for its efficacy in protection against DXR damage.

Thus, it is contemplated that other copper lowering agents find use to mimic TM's effect on protecting against DXR toxicity. Zinc is a copper lowering agent, as shown by its extensive use and effectiveness in Wilson's disease, a disease of copper toxicity, and also as shown here, by its ability to lower Cp levels in mice. Cp levels were lowered to the same degree at the start of the DXR experiment by zinc as with TM (FIG. 1). Zinc's effect were attributed to the high circulating zinc levels inducing intestinal cell metallothionein, which then blocks copper absorption from the diet, and reabsorption of endogenously secreted copper. While zinc protected significantly against DXR cardiac damage, as reflected in plasma LDH levels, it was overall less effective than TM. Thus, while zinc protection was significant in terms of LDH levels and equal to TM (FIG. 4), it showed no effect on CK MB (FIG. 3), and showed a lesser effect than TM and statistically insignificant effect on troponin I (FIG. 2).

The present invention is not limited to a particular mechanism. Indeed, an understanding of the mechanism is not necessary to practice the present invention. Nonetheless, in considering why these differences might occur, it is useful to consider the drugs' differences in mechanism of action. TM blocks absorption of copper, but also has a potent free copper complexing action when absorbed into the blood stream. Zinc, on the other hand, primarily blocks the intestinal absorption of copper. It is contemplated that DXR causes release of some copper from damaged tissue, which is involved in causing further damage to the heart. This copper is readily complexed by TM, but not by zinc. Theoretically this copper could be complexed by metallothionein induced by zinc in various tissues, but that metallothionein might not be as readily available to complex copper as TM. In this manner the protective effect of zinc might be less. This mechanism may also explain the greater increase in Cp levels at day 4 in zinc treated animals compared to TM treated animals (FIG. 1).

It has previously been found that zinc attenuates doxorubicin toxicity in mice. This effect, together with studies of another metallothionein inducer, bismuth, has been attributed to induction of metallothionein, and its scavenging of free radicals. Irrespective of the mechanism of zinc protection against DXR toxicity, it is clear that it is not as effective at protection as TM at the same level of copper depletion.

In summary, it was shown that the protective effect of TM against DXR cardiac toxicity is due to lowering availability of copper, since copper supplementation eliminated the effect. Further, it was shown that similar levels of systemic copper depletion using zinc or trientine, as assessed by serum Cp levels, is less effective in protecting against DXR toxicity than TM. The present invention is not limited to a particular mechanism. Indeed, an understanding of the mechanism is not necessary to practice the present invention. Nonetheless, it is contemplated that the differences in TM and zinc effects are due to their different mechanisms of action, in which TM complexes copper already in the body, and zinc does not. In it further contemplated that the differences between TM and trientine may be due to the differences in free copper binding by the two molecules.

All publications and patents mentioned in the above specification are herein incorporated by reference. Various modifications and variations of the described method and system of the invention will be apparent to those skilled in the art without departing from the scope and spirit of the invention. Although the invention has been described in connection with specific preferred embodiments, it should be understood that the invention as claimed should not be unduly limited to such specific embodiments. Indeed, various modifications of the described modes for carrying out the invention which are obvious to those skilled in the relevant art are intended to be within the scope of the following claims.

We claim:

1. A method of treating or preventing anthracycline induced myocardial fibrosis comprising administering a copper lowering agent to a subject under conditions such that said anthracycline induced myocardial fibrosis is prevented or reduced.

2. The method of claim 1, wherein said copper lowering agent is a thiomolybdate.

3. The method of claim 2, wherein said thiomolybdate is tetrathiomolybdate.

4. The method of claim 1, wherein said copper lowering agent is selected from the group consisting of zinc, trientine, d-penicillamine, clioquinoil and tetrathiotungstate.

5. The method of claim 1, wherein said copper lowering agent is administered prior to said anthracycline.

6. The method of claim 1, wherein said copper lowering agent is administered contemporaneously with said anthracycline.

7. The method of claim 1, wherein said copper lowering agent is administered after said anthracycline.

8. The method of claim 1, wherein said copper lowering agent is administered orally or parenterally.

9. A method of treating or preventing non-ischemic or ischemic dilated cardiomyopathy having confirmed myocardial fibrosis, comprising administering a copper lowering agent under conditions such that symptoms of said dilated cardiomyopathy are prevented or reduced.

10. The method of claim 9, wherein said copper lowering agent is selected from the group consisting of tetrathiomolybdate, trientine and zinc.
